(19) United States
(12) Patent Application Publication
(10) Pub. No.: US 2015/0150728 A1
(43) Pub. Date: Jun. 4, 2015

(54) Orifice Occluding Inflated Device

(71) Applicant: Gideon Williams Duvall, Boca Raton, FL (US)

(72) Inventor: Gideon Williams Duvall, Boca Raton, FL (US)

(21) Appl. No.: 14/093,468

(22) Filed: Nov. 30, 2013

(51) Int. Cl. A61F 11/10

(52) U.S. Cl. CPC A61F 11/10

(57) ABSTRACT

An orifice occluding inflated device that includes a fluid reservoir system operable to store excess fluid and apply positive fluid pressure, an inflatable balloon system configured to receive a fluid and impinge upon the walls of an orifice thereby causing occlusion, a fluid flow management system that regulates fluid flow between the inflatable balloon system and fluid reservoir system, a complementary systems package configured to be in communicative contact with the occluded orifice, and a vent in fluid commutation between the inner and outer occluded orifice operable to regulate the passage of fluid.

### CROSS-REFERENCE TO RELATED APPLICATION

This application claims priority to U.S. Provisional Patent Application No. US 61/797,130 filed on Nov. 30, 2012, the entirety of which is incorporated by reference.

### FIELD OF THE INVENTION

The present invention deals with orifice occluding inflated devices that utilize fluid transfer between two reservoirs.

### BACKGROUND OF THE INVENTION

The present invention is applicable in inflatable orifice inserted devices. Over time, many devices have been disclosed that utilize an inflatable device inserted into a body cavity such as an ear canal as exemplified by patents: U.S. Pat. Nos. 3,602,654, 4,133,984, and 4,834,211. These devices are typically composed of a pumping means, fluid control means, and inflatable balloon. If scaled large enough, currently disclosed inflation and deflation techniques can be made operable requiring acceptably low levels of dexterity. However, in reality, a commercially successful device must comply with the size requirements dictated by the consumer. As illustrated in the applicable prior art, the multitude of currently disclosed inflation and deflation techniques require high levels of dexterity and/or impractically large actuation surfaces impeding consumer adoption. Thus, a need exists for an inflation and deflation system that can satisfy the size and dexterity requirements of the consumer thereby making inflatable in ear canal inserted devices viable consumer products. Furthermore, a successful device will be of sufficiently small size that it is able to be integrated with additional complementary technologies, such as microphones and speakers that take advantage of the benefits of ear canal occlusion provided by an inflatable device while still meeting the size and operational requirements of the consumer.

Along with meeting the size and dexterity requirements of the consumer, a successful device must also be readily and efficiently manufactured. The majority of currently disclosed orifice inserted devices rely on a valve that must be “zero leak” to ensure the device does not deflate prematurely. Due to the difficulties in manufacturing adequately small “zero leak” valves that have the appropriate flow characteristics and their associated cost, a new design for an inflatable orifice inserted device is needed that does not rely on a “zero leak” valve.

### SUMMARY OF THE INVENTION

The present invention relates to orifice inserted inflatable device that satisfies the outlined need, not relying on a “zero leak” valve for successful operation, is capable of being readily operated by a user and seamlessly integrated with complementary devices. An exemplary orifice inserted inflatable device is configured such that device deflation and/or inflation is operated by forces readily applied to distinctly different surfaces roughly the diameter of a human fingertip or larger regardless the physical size of the device's mechanical actuator. Additionally, the external prime actuation forces should be successfully applied in an inexact fashion with general directionality. Lastly, the device should be designed such that it will successfully operate with or without a user actuated flow control device or valve. An orifice inserted inflatable device satisfying these criteria may be coupled with a Complementary Systems Package (CSP) and in a manner that satisfies the size requirements of the consumer.

In order to achieve these three criteria, an exemplary system includes a Fluid Reservoir System (FRS), Fluid Flow Management System (FFMS) and an Inflatable Balloon System (IBS). The FRS is a fluid reservoir system which is configured to store excess fluid and apply positive fluid pressure to the IBS. The FFMS manages the fluid movement between the FRS and IBS. The IBS is an inflatable system that is in operable connection with the orifice and is configured to receive fluid and positive pressure from the FRS via the FFMS resulting in its inflation and preferable orifice occlusion.

Although the invention is illustrated and described herein as embodied in an orifice occluding inflated device it is not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 Illustrates an exemplary embodiment of the described invention inserted in a human ear.

FIG. 2 Illustrates a sectional view of a non-limiting embodiment of the present invention adapted for use in an ear canal in its initial or inactivated state.

FIG. 3 Illustrates a sectional view of a an alternate non-limiting embodiment of the present invention adapted for use in an ear canal in its initial or inactivated state.

FIG. 4 Illustrates a sectional view of the embodiment depicted in FIG. 2 in the deflated or activated state.

FIG. 5 illustrates a sectional view of the embodiment depicted in FIG. 2 inserted in human ear of FIG. 1.

FIG. 6 Illustrates an embodiment as shown in FIG. 4 in an orifice occluding state.

FIG. 7 Illustrates a sectional view of an alternate non-limiting embodiment of the present invention adapted for use in an ear canal in its initial or inactivated state.

FIG. 8 Illustrates a sectional view of the embodiment depicted in FIG. 7. in the deflated or activated state.

FIG. 9 Illustrates a sectional view of the embodiment depicted in FIG. 8 inserted in the human ear of FIG. 1.

FIG. 10 Illustrates the embodiment depicted in FIG. 9 in an orifice occluding state.

FIG. 11 Illustrates a isometric view of the embodiment depicted in FIG. 10.

FIG. 12 Illustrates a sectional view of an alternate non-limiting embodiment of the present invention coupled to a CSP adapted for use in an ear canal in the device's initial or inactivated state.

FIG. 13 Illustrates an sectional view of a the embodiment depicted in FIG. 12 in its deflated or activated state.

FIG. 14 Illustrates a top isometric view of the embodiment depicted in FIG. 12.

FIG. 15 Illustrates a bottom isometric view of the embodiment depicted in FIG. 12.

FIG. 16 Illustrates an exploded top isometric view of the embodiment depicted in FIG. 12.

FIG. 17 Illustrates an exploded bottom isometric view of the embodiment depicted in FIG. 12.

FIG. 18 Illustrates an alternate embodiment of the device depicted in FIG. 12.

FIG. 19 Illustrates device of FIG. 18 in an alternate state.

FIG. 20 Depicts an isometric bottom view of the embodiment depicted in FIG. 18.

FIG. 21 Illustrates a cross sectional view of an alternate non-limiting embodiment of the present invention adapted for use in an ear canal in its initial or inactivated state.

FIG. 22 Illustrates an cross sectional view of the embodiment depicted in FIG. 21 in its deflated or activated state.

FIG. 23 Illustrates an cross sectional view of the embodiment depicted in FIG. 21 coupled to an exemplary CSP.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms. Although the invention is illustrated and described herein with reference to specific embodiments, the invention is not intended to be limited to the details shown. Rather, various modifications may be made in the details within the scope and range of equivalents of the claims and without departing from the invention.

The disclosed invention is composed of three primary subsystems; the Inflatable Balloon

System (IBS) Fluid Reservoir System (FRS), and the Fluid Flow Management System (FFMS).

### IBS Description

The Inflatable Balloon System (IBS) is in fluid connection to both the Fluid Reservoir System (FRS) and Fluid Flow Management System (FFMS) with the IBS supplying fluid to the FRS via the FFMS during device deflation and the FRS supplying fluid to the IBS via the FFMS during inflation. In practice, the IBS consists of an inflatable balloon which is designed to impinge upon the walls of an orifice such as an ear canal creating a pneumatic seal. Preferably the IBS is composed of an inelastic or non-compliant balloon however a compliant balloon or combination of compliant and non-compliant materials may also be utilized. Exemplary materials include but are not limited to high durometer (70-120 A) TPU, PET, Pebax and Nylon. Alternately, the inflatable portion of the IBS may be comprised of an elastic membrane conventionally referred to as a compliant balloon; exemplary materials would be latex, TPU (low durometer<70 A), or silicone. Additionally, a hybrid system may be utilized composed of a non-compliant balloon with a compliant balloon on top. In the later embodiment, the fluid is contained within the underlying non-compliant balloon with the compliant balloon used to provide compression, improve esthetics, and modify the acoustical characteristics of the system. In a hybrid system, an appropriate bio compatible lubricant should be utilized to minimize the friction between compliant and non-compliant balloons thereby minimizing inflation pressure. Examples of an applicable lubricant would be fluorosilicone oil and teflon used with a TPU non-compliant balloon and silicone rubber compliant balloon.

An alternate configuration of the hybrid system may be composed of a balloon of the compliant or non-compliant type or a combination thereof with an exterior foam layer. An exemplary configuration would be a compliant foam composed of polyurethane or silicone with a thickness of between 0.01 inches and 0.05 inches configured to act as an interface between the underlying balloon and the orifice wall. Preferably the foam layer is configured such that it is in its static, unstressed state when the balloon is in its fully expanded state. While it is preferable that the said foam layer is mechanically coupled to the underlying balloon membrane, it may also exist as a un-mechanically coupled layer. Said foam layer may serve several purposes, including but not limited to the modification of the acoustical properties of the overall device, increase device durability, and improved user comfort.

As will be understood by those skilled in the art, when determining the optimal balloon material, chemical compatibility, biocompatibility, permeability and system inflation pressure should be of primary concern. As previously described, the present invention utilizes the pressure within the FRS to inflate the IBS and impinge on the orifice walls. Thus it is of prime importance to minimize the pressure required to fully inflate the IBS and match this inflation pressure with the FRS fluid storage pressure as it will need to provide a pressure equal to or greater than that required to fully inflate the IBS and impinge on the orifice walls to create an occluding seal. Depending on the orifice and IBS material, an optimal orifice wall impinging pressure is between 0.25-4.0 psi. Thus an IBS balloon material should be selected in conjunction with the FRS design such that the system is able to provide the aforementioned pressures on the orifice wall.

A preferred IBS configuration utilizes a non-compliant balloon as it reaches full inflation before internally pressurizing. There are several advantages such an IBS balloon material offers, primarily, the IBS balloon is able to conform to irregular shaped orifices and provide uniform pressure on orifice walls optimizing user comfort. Additionally, a non-compliant balloon expands without building internal pressure until reaching full inflation, therefore, pressure provided by the FRS will be equivalent to the pressure exerted on orifice walls. This minimizes the amount of pressure required from the FRS making the system more robust. Finally the use of a non-compliant balloon mitigates the permeability problems associated with sealed liquid filled balloon based systems as the disclosed system will constantly be in a state of full inflation or under positive pressure thus mitigating the problems associated with gas diffusion into the system. In the case of a gas filled system, the leakage rate of gas through the device can be made sufficiently low, however, gas loss while in use will be unavoidable due to the positive internal device pressure. The use of a non-compliant balloon enables the device to regain lost gas when not in use as changes in barometric pressure in its environment will create a pressure differential across the IBS membrane with its interior being lower pressure than outside. Diffusion of gas into the balloon will occur until the IBS regains its fully inflated state.

### FFMS Description

In operable connection to the IBS is the FFMS which is also in operable connection to the FRS. The FFMS facilitates fluid flow between the IBS and FRS. The FFMS is preferably designed to provide unequal flow rates in the forward and reverse direction which may or may not be modulated by an external applied force. In practice the FFMS is ideally configured to facilitate fluid flow from the IBS into the FRS at a rate substantially higher than the rate of flow from the FRS into the IBS. This rate difference enables the user to prepare the device for installation rapidly with fast flow of fluid from the IBS into the FRS. The slower rate of flow in the reverse direction, FRS to IBS, preferably provides the user with ample time to position the device in the orifice before the IBS inflates to an orifice occluding diameter.

An alternate configuration of the FFMS utilizes a valve that is essentially a check valve with the ability to be selectively opened by the application of a force from a prime mover enabling flow in the checked direction. An exemplary valve would be a duck bill valve, which is ideally designed to provide high rates of flow at low pressures in one direction and no flow (in reality extremely slow flow) in the reverse direction. Additionally, it is well known by those skilled in the art that correctly applied pressure from a prime mover can result in deformation and opening of the duckbill valve enabling flow in the reverse or checked direction. In practice, this configuration affords the user an indefinite period of time to install the device within the orifice. Once situated, the user can rapidly inflate the IBS by applying a force from a prime mover, such as their finger, to the valve, enabling the flow of fluid in the reverse direction causing IBS inflation and orifice occlusion. As the said valve will be positioned within the sealed device, the prime actuating force will preferably be applied to and transferred through a wall of the device which may or may not be uniquely adapted for this purpose. In order to meet the demands of the consumer, the disclosed device will need to be made as diminutive as possible, thus, insufficient exposed surfaces may exist to incorporate a valve actuation surface of sufficient size to be readily operated directly by a prime mover such as the user's finger. Thus, an alternate actuation method may be employed which utilizes the tissue surrounding the said orifice inserted device to transfer the actuation force applied by the prime mover to the valve actuation surface. An exemplary configuration of the aforementioned valve actuation system may be integrated into a device designed to be utilized for the occlusion of an ear canal. In this instance, the IBS is configured to be in operable connection with the user's ear canal, FFMS is in operable connection with the tragus and the FRS is positioned in the concha. In use, the user initially deflates the IBS by applying force on the IBS, transferring fluid into the FRS. Then the device is inserted into the user's ear canal such that the IBS is in operable connection with the ear canal and the FFMS valve actuation surface is in operable connection with the tragus. Once the user desires to inflate the IBS, he applies force from a prime mover, such as their finger, onto the tragus and/or the tissue immediately adjacent to the tragus, resulting in its deformation and the deformation of the device's valve actuation surface in operable connection, actuating the FFMS valve into an open position enabling the flow of fluid into the IBS resulting in its inflation and ultimate occlusion of the ear canal.

As will be obvious, FFMS may utilize a multitude of different styles of valves that can configured to satisfy the aforementioned operational criteria such as duckbill, poppet, flapper, umbrella, etcetera, or a combination therein, as long as the aforementioned operation is achieved. Utilizing tissue in operable connection to the FFMS actuation surface to transfer the actuating force enables the actuating surface to be made smaller than the size of the prime mover and positioned such that it is not fully exposed to the prime mover thus facilitating miniaturization of the overall inflatable ear canal inserted device.

### FRS Description

The FRS is in operable connection to the FFMS and IBS. It receives and stores fluid from the IBS during its deflation and supplies the fluid and driving force for the movement of fluid through the FFMS back into the IBS and the pressure/force on the orifice walls which creates the occluding seal. There are a multitude of ways to configure the said FRS, however, in its most ideal theoretical manifestation the FRS is a fluid reservoir that is of minimal size and stores the fluid under a constant pressure regardless of the contained volume with no fluid loss and minimal impedance.

In practice, the FRS may be composed of an expanding elastic membrane, spring loaded piston-cylinder system or the like to accommodate the fluid from the IBS and store the energy provided by the user during IBS deflation which ultimately is used to re-inflate the IBS and provide occluding pressure on the orifice walls. Regardless its construction, its effective spring constant should be matched to the IBS inflation pressure to ensure that in use, the IBS exerts between 0.25 and 4.0 psi on the orifice walls. As will be obvious to those skilled in the art, the FRS should utilize materials that have exceptionally good compression set or minimal loss of potential energy when in use. Exemplary energy storage material would include but not be limited to the class of silicone rubbers and metallic springs.

The disclosed orifice inserted inflated device is a closed system which can be filled with either liquid or gasses. In the case of gas, it may be filled with a multitude of gasses, however, in reality, the unpreventable diffusion of gas through the device makes the use of any gas other than air less than practical. In the case of a liquid, the liquid will ideally possess certain qualities that are optimal for mechanical, electrical, biological safety, and acoustic properties. With regards to mechanical/physical properties, the liquid should have a viscosity below 10 cp, possess a pour point below −20° C., boil at a temperature above 80° C., be non-flammable, and have a low specific heat. With regards to biological safety, the liquid must be biologically compatible, evaporate from human skin leaving no residue in less than 24 hours and be non-flammable. Electrically, the liquid should be a dielectric such that any rupture of the FRS, FFMS, or IBS will not have a permanent negative impact on the electrical performance of adjacent electronics. Additionally, a dielectric fluid will prevent potentially dangerous transfer of electrical energy to tissue in operable connection in the event of a fluid leak. When the device is used to occlude and provide acoustical attenuation to an ear canal, the higher the liquid density, the higher the attenuation. Exemplary liquids are the class of liquids known as fluorocarbons. Depending on the molecular structure, the physical properties of these fluids varies; however, the vast majority of distinct fluids within the fluorocarbon family possess the aforementioned qualities and have been heretofore not enumerated by prior art. An exemplary fluid is FC-770 manufactured by 3M. As the system is closed, preventing diffusion of fluid into and out of the device is critical, thus the FRS should be designed such that the overall system is of ultra-low permeability.

Alternately, the device may be filled with a liquid which may turn from a liquid to a non-liquid. An exemplary system would include a two part silicone gel/rubber system where one part may initially be contained in the IBS and the other part may be initially contained in the FRS with the two components appreciably separated by the FFMS. In use, the user deflates the IBS thus transferring its liquid component into FFMS such that the IBS enters into its deflated state and both components of the two part silicone gel system are now combined within the FRS and are now considered activated. Upon the removal of the user's deflation force, the activated fluid then flows from the FRS through the FFMS and into the IBS as previously described. The activated fluid then changes state from a liquid to a gel or rubber. As will be obvious, the time over which the activated fluid solidifies, or cure time, should be kept appreciably long as to enable the device to be inserted by the user into the desired orifice.

Metallic or non-metallic particles such as Tungsten or glass microspheres powder may be included within the aforementioned liquids utilized within the device for a multitude of purposes including but not limited to the blockage of electromagnetic radiation and/or the modification of mechanical transmission of vibrations through the device.

During device removal, the occluding seal may produce a vacuum within the sealed orifice. In the case of an ear canal, it is advantageous to minimize and/or eliminate the creation of a vacuum during the extraction of the present invention. In order to accomplish this, a conduit that is in operable connection with the interior portion of the sealed orifice and the ambient atmosphere may be incorporated into the device. Acting as a vent, the conduit facilitates the displacement of any vacuum when the device is removed. As it may be found disadvantageous to have said vent open on a continuous basis, the vent may be in operable connection with a valve, rather than the ambient atmosphere. A valve, in operable connection with the ambient atmosphere and vent conduit, may passively enable the mitigation of a pressure differential or may be configured to open when the user pulls on the device during removal.

As will be obvious to those skilled in the art, the disclosed device may be coupled with a CSP which takes advantage of the unique abilities and attributes of the disclosed invention. However, the present invention discloses two unique configurations which have been found to offer unique ability to result in rapid integration of the technology into the mass market.

The first configuration is that which is designed to be adapted to existing non-customized CSP, such as, ear buds, earphones or hearing aids. An example of this would include the development of a product to replace ear bud style foam or silicone tips. An alternate example would be the integration of the invention onto the Receiver in the canal of a Receiver In Canal hearing aid. The second configuration are products which are uniquely designed to couple a CSP to the disclosed orifice inflated device; examples of this are disclosed below.

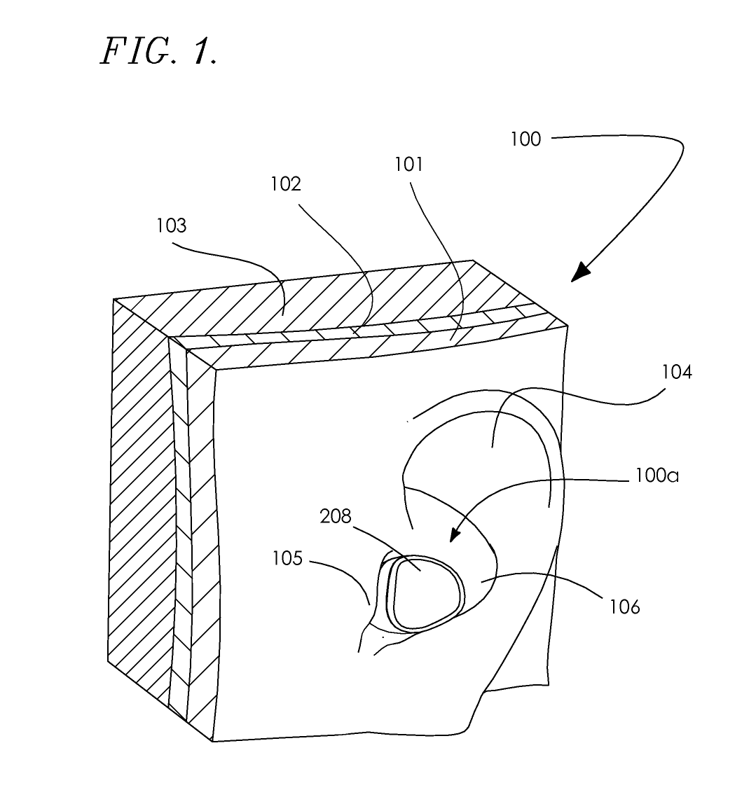

Referring to FIG. 1, an exemplary embodiment of the described invention is shown inserted in a human ear. Ear region 100 depicts a section of the human head that includes the ear. Ear region 100 depicts inner skull region 103, skull bone 102, outer soft tissue 101 and ear pinna 104. Orifice inserted inflated device 100a is depicted inserted in operable connection with ear canal (not shown), concha 106 and tragus 105 with FRS reservoir elastic membrane 208 shown.

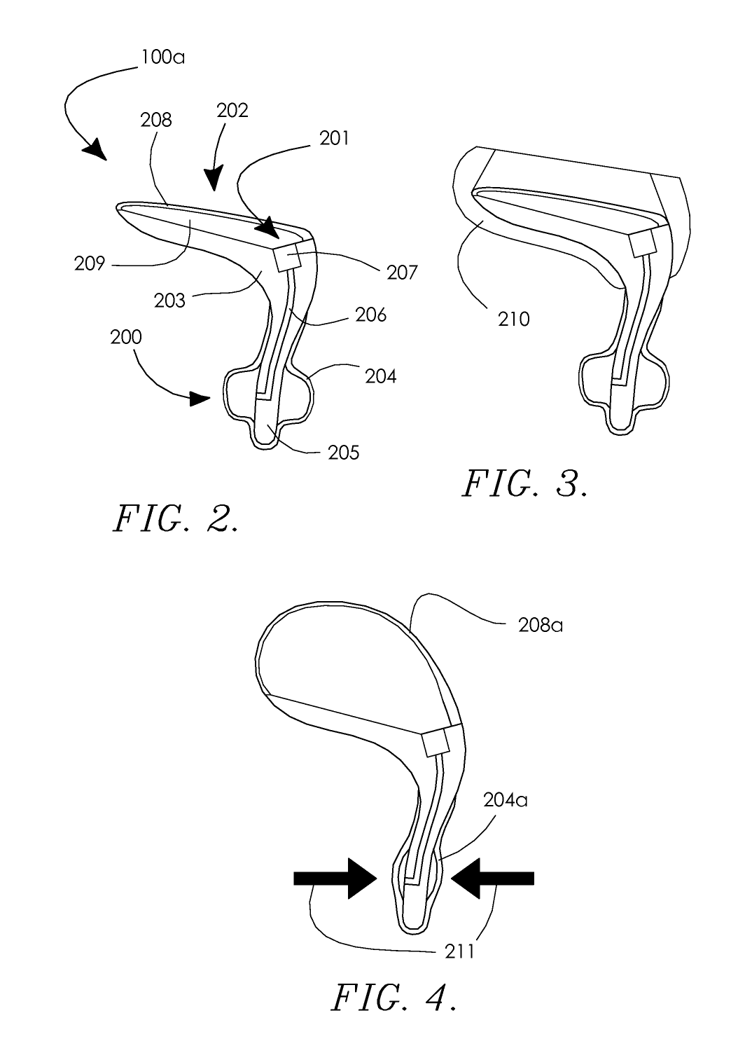

Referring to FIG. 2, the device depicted is an exemplary non-limiting embodiment of the orifice inserted inflated device 100a adapted for use in a human ear canal. It is composed of three primary systems, IBS 200, FFMS 201, and FRS 202 all in operable connection and integrated into one device body 203. Depending on the design requirements, device body 203 may be composed of multiple materials and multiple components, however, in this embodiment, it is formed as one. Regardless of the number of materials utilized, optimal materials are biocompatible, durable, have good compression set and are sufficiently soft (typically between 35 and 60 durometer Shore A). As the present invention is a sealed system, low permeability of device body 203 is of high importance, thus an optimal material will be of exceptionally low permeability. An exemplary elastomer is butyl rubber. Alternately, the device body may be configured to contain an interior lining or exterior coating which possesses sufficiently low permeability enabling the gross portion of the device body 203 to be made from an alternate elastomer, such as TPE or silicone which may offer advantages with respect to cost or user experience.

The IBS 200 is composed of balloon system 204, which may be composed of a single balloon of the compliant or non-compliant variety or alternately a hybrid system both of which may have an exterior coating of foam as previously described. Balloon system 204 is preferably bonded to shaft 205 which is in operable connection to FFMS 201 via conduit 206. As depicted in FIG. 2, shaft 205 and conduit 206 may be incorporated in device body 203.

FFMS 201 is preferably composed of flow regulation device 207 which facilitates fluid flow between IBS 200 and FRS 202. As previously described, the flow regulation device preferably facilitates fluid flow from the IBS 200 into FRS 202 at a substantially higher rate than it does in the reverse direction under an equivalent driving force. While not ideal, a system may be designed with a flow regulation device 207 or an operational equivalent that has equal flow in both directions.

As previously described, FRS 202 may be composed of multiple operational equivalents, however, in the depicted embodiment, FRS 202 is composed of FRS elastomeric membrane 208 which is in operable connection with fluid reservoir 209 and is uniquely configured to be the sole component of FRS 202 that deforms in order to accommodate fluid transferred from the IBS and store potential energy. As previously described, FRS elastomeric membrane 208 ideally has no compression set and is configured to provide fluid to the IBS at a pressure which results in an actual force of between 4.0 and 0.25 psi on the orifice walls when occluding an orifice or ear canal. Exemplary elastomeric materials would include elastomers such as silicone or latex.

The device depicted in FIG. 3 is an alternate non-limiting embodiment of orifice inserted inflated device 100a depicted in FIG. 2. In this embodiment IBS 200 is identical to that of the embodiment depicted in FIG. 2, additionally, the function and operation of FRS 202 and FFMS 201 are identical. However, an exterior material 210 may be applied to improve the user experience and/or comfort. Exemplary materials would include foams made of urethane or silicone.

FIG. 4 depicts the embodiment depicted in FIG. 2 in an activated or deflated state. When the user wishes to install the device in an orifice, first, IBS 200 is deflated by applying pressure onto balloon system 204 of IBS 200 with IBS deflation force 211 of sufficient magnitude to facilitate the transfer of fluid through FFMS 201 into FRS 202 resulting in the stretching of FRS elastomeric membrane 208 into stretched FRS elastomeric membrane 208a, pressurization of the FRS and the transition of balloon system 204 into deflated balloon system 204a.

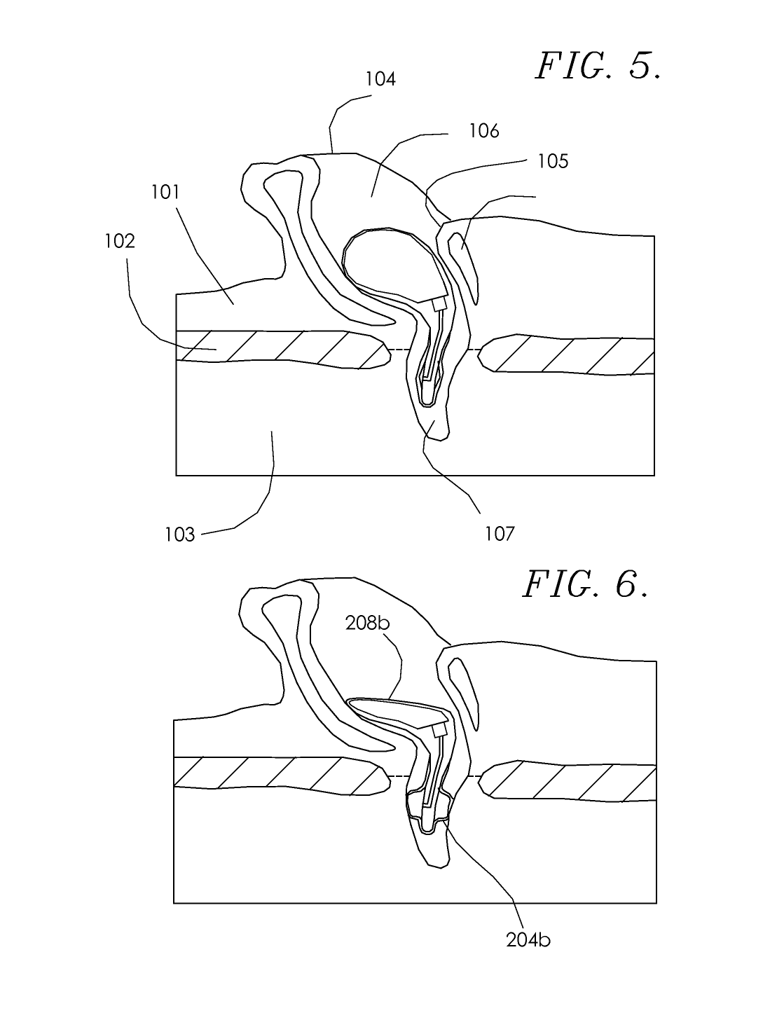

Referring to FIG. 5, once IBS 200 is sufficiently deflated, the user removes IBS deflation force 211, inserts orifice inserted inflated device 100a into an orifice, such as an ear canal, whereby the IBS 200 is in operable connection with ear canal 107. In this exemplary embodiment, once the user releases IBS deflation force 211, a pressure differential now exists across FFMS 201 with the FRS 202 having a higher internal pressure than the IBS 200, thus, fluid flows back into the IBS with the flow rate managed by flow regulation device 207. As re-inflation of IBS 200 will begin instantaneously, flow regulation device 207 of FFMS 201 is preferably configured to provide sufficient time to situate the device in the ear canal before IBS 200 re-inflates beyond an occluding diameter. Depending on the device configuration the optimal re-inflation time will vary, however, generally, a full re-inflation time of 30-60 seconds is recommended.

Referring to FIG. 6, balloon system 204 inflates to a diameter where it impinges on walls of ear canal 107, it enters into occluding balloon system 204b state and the pressure inside IBS 200 will equilibrate with that inside FRS 202. The pressure exerted on the walls of the ear canal 107 is equal to the IBS 200 internal pressure when balloon system 204 contacts the walls of ear canal 107 and that of IBS 200 after equilibrating with FRS 202. This pressure differential, and thus the pressure on the ear canal walls, should be kept between 0.25 and 4.0 psi. Once steady state pressure in IBS 200 and FRS 202 is reached, FRS elastomeric membrane 208 enters into state, occluding FRS elastomeric membrane 208b.

When the user desires to break the occluding seal and remove orifice inserted inflated device 100a, the user gently pulls on the device, breaking the occluding seal and removing the device. As discussed in the previous description and as is depicted and discussed in later figures, valves may be included in orifice inserted inflated device 100a that are in operable connection with the ambient field and sealed orifice which, during the natural course of removal, open, mitigating any pressure differential that may be generated by balloon system 204 during removal.

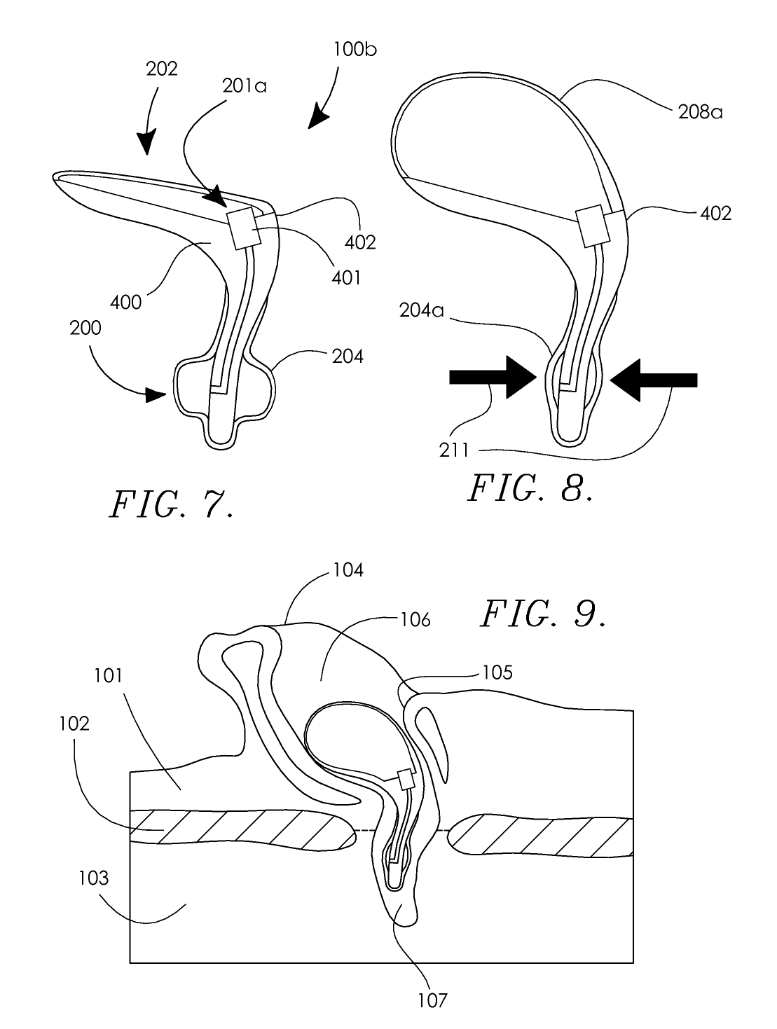

An alternate embodiment of the present invention is depicted in FIG. 7. In this embodiment, user actuated orifice inserted inflated device 100b, IBS 200 and FRS 202 are operational equivalents to those described in FIG. 2. Unique to this embodiment is FFMS 201a which is composed of user actuated valve 401 and valve actuation surface 402 which is in operable connection and preferably incorporated within actuated device body 400. User actuated valve 401 is configured to operate as a check valve with a manually actuated release. In use, user actuated valve 401 is configured to provide minimally impeded fluid flow from IBS 200 into FRS 202 and prevent flow in the reverse direction. Upon receiving an external force from a prime mover user actuated valve 401 enters into an actuated state enabling flow in the reverse or checked direction from FRS 202 into IBS 200. Preferably user actuated valve has a minimal forward crack pressure of between 0.0 and 1.0 psi and a minimum forward flow rate of 1 cc/s with a 1 cp fluid with a pressure differential of between 1-2 psi. As will be obvious, the creation of a zero leak valve at these proportions and performance criteria and back pressures is difficult, however, the design of the present invention enables the device to operate successfully if user actuated valve 401 is not “zero” leak, thus enabling the use of a multitude of low cost pre-existing valve solutions. In the event that user actuated valve 401 is not “zero” leak, FFMS 201a will operate in a similar fashion to FFMS 201, however, the user will be able to rapidly inflate balloon system 204 once the device is positioned in the appropriate orifice by manually actuating user actuated valve 401. In order to transition user actuated valve 401 into the actuated state, it is preferably configured to be in operable connection with external valve actuation surface 402 which is configured to receive a unique actuation force either directly or indirectly from a prime mover, such as a user's finger, and transfer this force to user actuated valve 401 causing it to enter into its actuated state thus facilitating fluid flow in the reverse direction. Upon removal of a prime actuation force, user actuated valve 401 returns to its un-actuated state preventing flow in the checked direction.

FIG. 8 depicts the transition of the device depicted in FIG. 7 into its deflated/activated state in preparation for insertion into an orifice. This process is accomplished in an identical fashion to that previously described for the embodiment of FIG. 2 with minimally impeded fluid flow from IBS 200 into FRS 202 in response to the pressured differential created by IBS deflation force 211 applied to balloon system 204. This force causes balloon system 204 to deflate thus becoming deflated balloon system 204a and FRS elastomeric membrane 208 to become stretched FRS elastomeric membrane 208a. However, unlike FFMS 201, FFMS 201a is configured to prevent flow in the reverse direction. Thus, this embodiment will ideally stay in this activated/deflated state for an indefinite period of time affording the user indefinite time for insertion into the orifice. In practice, FFMS 201a will likely leak in the checked direction, thus an ideal system will be configured such that the leakage will fully re-inflate IBS 200 in no less than 60-120 seconds. As will be obvious to those skilled in the art, actuated device body 400 and external valve actuation surface 402 will be configured to not actuate user actuated valve 401 during the course of IBS 200 deflation, corresponding FRS 202 inflation, or general device usage.

Once actuated orifice inserted inflated device 100b has been activated/deflated, the user then inserts the device in the appropriate orifice such as ear canal 107 as depicted in FIG. 9 with IBS 200 in operable connection with ear canal 107. As depicted, FFMS 201a is configured to be actuated through the application of force from a prime mover applied to tissue adjacent to tragus 105 and transferred to valve actuation surface 402 through the tissue in operable connection. Therefore, valve actuation surface 402 of actuated orifice inserted inflated device 100b is situated in operable connection with tragus 105.

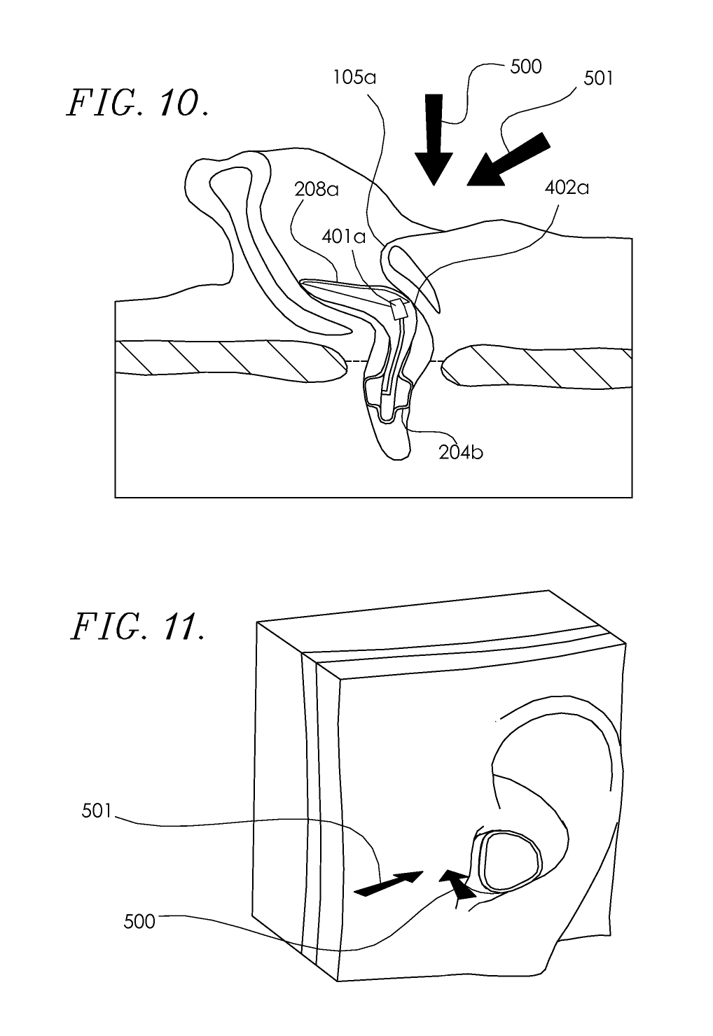

Referring to FIG. 10 and FIG. 11, when the user desires to inflate balloon system 204 to cause occlusion of ear canal 107, the user applies force such as normal force 500 or sheer force 501 from a prime mover, such as a finger, onto the tissue in operable connection with tragus 105 causing its deformation into deformed tragus 105a. Deformed tragus 105a transfers actuation forces such as normal force 500 and sheer force 501, depicted in FIGS. 5a and 5b, to valve actuation surface 402 causing it to deform into actuated valve actuation surface 402a ultimately activating user actuated valve 401 causing it to enter into actuated user actuated valve 401a thereby enabling flow of fluid from the FRS 202 into IBS 200 ultimately inflating balloon system 204 into occluding balloon system 204b sealing ear canal 107. Once sufficient level of inflation has been achieved, user removes actuation force and valve 402 enters into its static back flow preventing state. Removal of actuated orifice inserted inflated device 100b is accomplished in an identical fashion as orifice inserted inflated device 100a.

As previously discussed, the aforementioned orifice sealing devices may be coupled to a CSP to increase the functionality of overall device. Depicted in FIG. 12 through FIG. 17 is an exemplary embodiment of how a CSP may be designed to be coupled with actuated orifice inserted inflated device 100b or orifice inserted inflated device 100a.

FIG. 12 depicts a cross sectional view of CSP—actuated orifice inserted inflated device 100c which is actuated orifice inserted inflated device 100b adapted to contain CSP 610. IBS 200, FFMS 201a and FRS 202 of CSP—actuated orifice inserted inflated device 100c are operational equivalents to those of actuated orifice inserted inflated device 100b. As will be obvious in light of FIG. 12 and FIG. 7, this embodiment contains CSP 610 within CSP coupled actuated device body 601 which is actuated device body 400 configured to contain CSP 610 and facilitate the functionality of its contained transducers. Similarly, valve actuation surface 604 operates in an identical fashion to valve actuation surface 402. Unique to CSP coupled actuated device body 601 is CSP retention flap 602 which retains CSP 610. CSP 610 may contain transducers which rely on pneumatic pressure for their operation, FIG. 12 depicts CSP 610 in operable connection with the space formed by IBS 200 via conduit 603 which is molded into CSP coupled actuated device body 601. In order to ensure a pneumatic seal between CSP 610 and conduit 603, CSP 610 may include protruding portion 611 which operates as a “hose barb” coupling conduit 603 to CSP 610. As will be obvious, more than one conduit may be included into CSP coupled actuated device body 601 to meet the needs of CSP 610. CSP 610 may be designed to be in physical operable connection to an external system via external conduits such as tubes, wires or the like. In order to facilitate this external connection CSP external interface 612 may be configured to be in operable connection with CSP 610.

As will be understood by those skilled in the art, the ability to readily couple transducers, such as microphones and speakers to the sealed, occluded space created by IBS 200 in its occluding state and position transducers, such as microphones or accelerometers proximal the orifice has myriad advantages. For instance CSP—actuated orifice inserted inflated device 100c may be configured to operate as a ear bud thus CSP 610 may include a speaker which is in operable connection with sealed air space formed by inflated IBS 200 via a conduit such as conduit 603. Such a device will be able to transmit sound/pneumatic pressure waves to the user's tympanic membrane more efficiently, thereby improving the user's listening experience. Additionally, the occluding seal created by IBS 200 may attenuate the ambient sound field of the user which may enable the user to listen to their music at lower sound pressure levels while experiencing the same perceived volume. A further benefit to the user may be derived from the stabilization afforded by IBS 200 in its occluded state which improves stability of CSP 610 in operable connection.

FIG. 13 depicts the transition of the device depicted in FIG. 12 into its activated/deflated state in preparation for insertion into an orifice. This process is accomplished in an identical fashion to that previously described for the embodiment of FIG. 7 and depicted in FIG. 8.

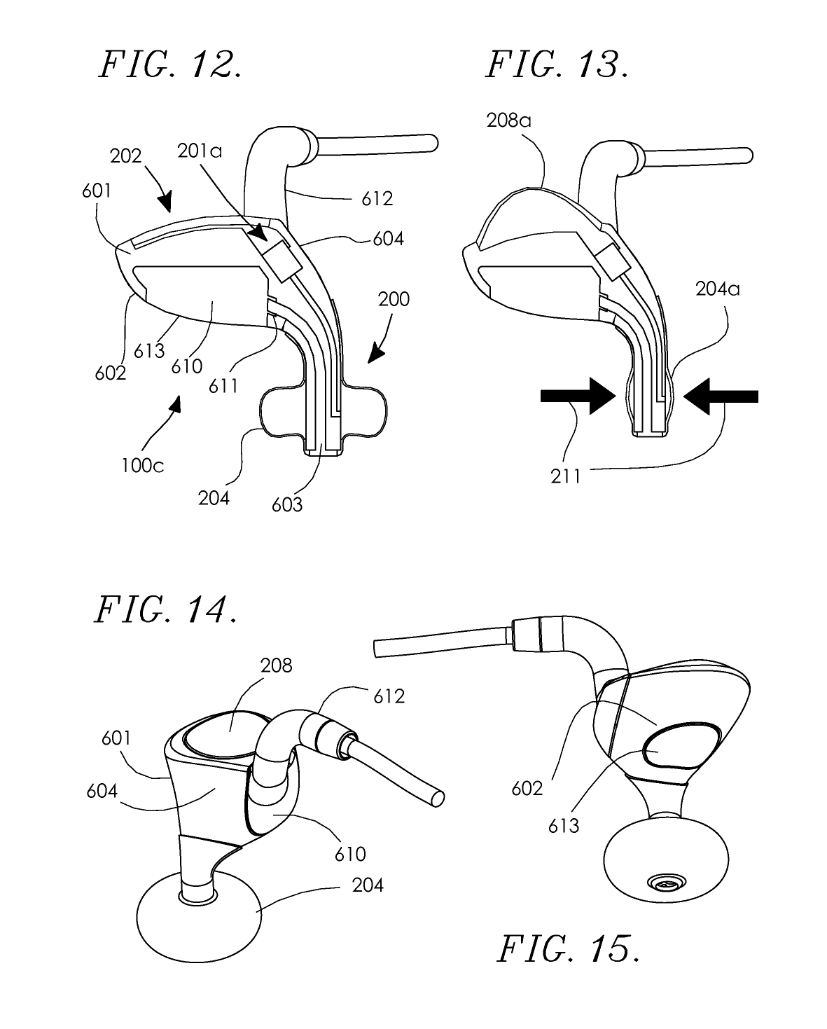

There are a multitude of ways to securely couple a CSP with an orifice inserted inflated device body. Depicted in FIG. 14, FIG. 15, FIG. 16, and FIG. 17 is an embodiment of CSP—actuated orifice inserted inflated device 100c and a single methodology which is found to be exemplary. In this embodiment, CSP 610 is inserted into the CSP pocket 614, depicted in FIG. 16, of CSP coupled actuated device body 601 and held in place by an interference fit facilitated by the elastic material of CSP retention flap 602. As depicted in the figures, it is preferable for CSP 610 to be positioned proximal the orifice and its adjacent tissue/material with FRS 202 positioned less proximal thereby ensuring that CSP 610 does not interfere with the operation of FRS 202. As will be obvious, the amount of interference and/or retention force provided by CSP retention flap 602 utilized to hold the CSP 610 within device body 601 will be a function of many factors including modulus of CSP coupled actuated device body 601 material, wall thickness and size of CSP retention flap 602, and size of undercut when CSP 610 is installed in device body 601. However, ideally greater than half of CSP 610 will be contained within CSP retention flap 602 such that mechanical interference from an “undercut” may be present. CSP 610 may be designed to be in physical operable connection to an external system via external conduits such as tubes, wires or the like. In order to facilitate this external connection CSP external interface 612 may be configured to be in operable connection with CSP 610.

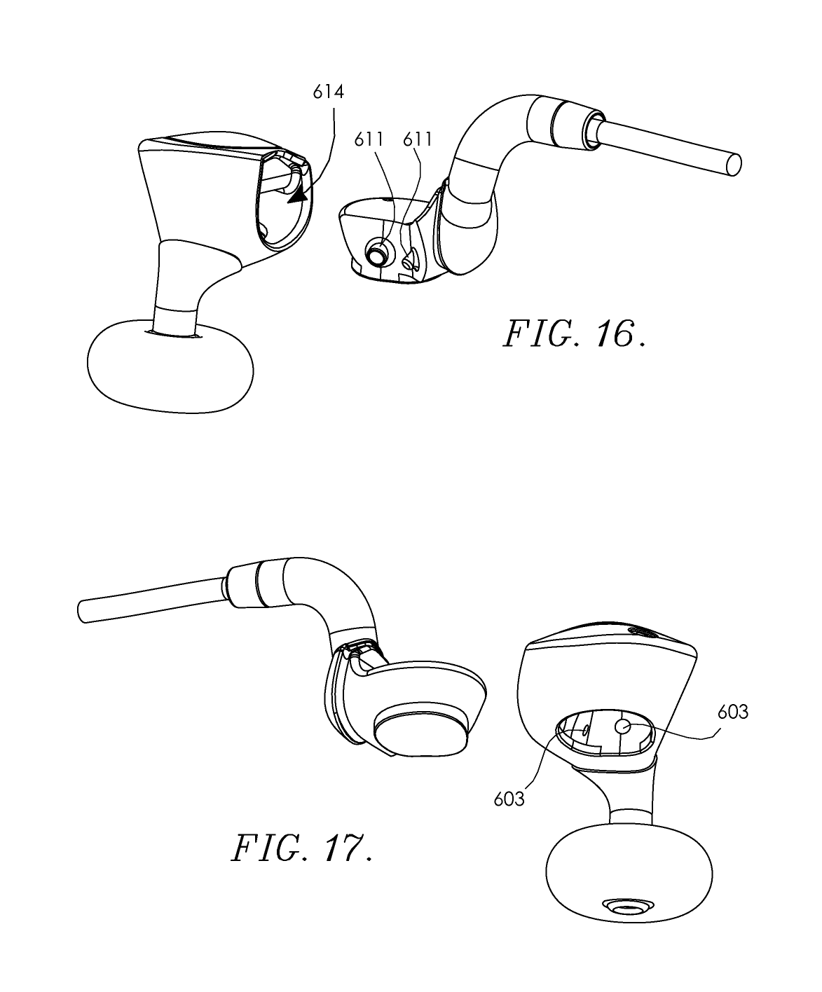

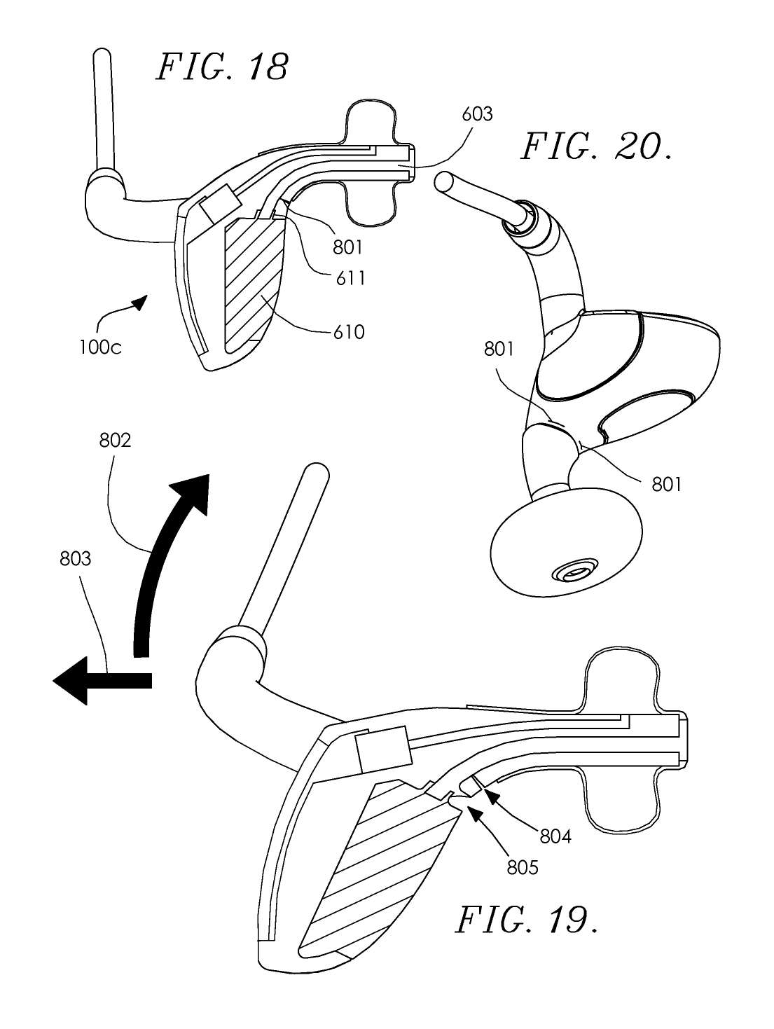

In the depicted embodiment, CSP 610 possesses CSP protrusion 613 which is preferably protrudes from the bottom of CSP 610 in a fashion which enables it to be completely surrounded by CSP retention flap 602 as depicted in the embodiment. As will be obvious, CSP protrusion may or may not be included in CSP 610. CSP protrusion 613 has three functions. First, it increases the available internal volume of CSP 610 which may be utilized to improve functionality. Secondly, CSP protrusion 613 may be utilized to increase amount of mechanical interference between CSP 610 and CSP coupled actuated device body 601 decreasing the chance of unintended disconnection between the two entities. Lastly, as will be explained in further detail in FIGS. 18, 19, and 20, CSP protrusion 613 may be configured to facilitate the breaking of the pneumatic seal of an orifice created by IBS 200 upon intended or unintended removal of CSP—actuated orifice inserted inflated device 100c.

FIGS. 18, 19, and 20 depict two systems whereby the seal created by IBS 200 may be affectively broken in a manner other than through the disconnection of balloon system 204 with orifice walls. Such a system may be advantageous during the intended or unintended removal of the disclosed embodiments of the present invention. As will be obvious to those skilled in the art, these techniques are described in relation to CSP—actuated orifice inserted inflated device 100c, however, they may be adapted to other embodiments or be composed of alternate equivalent systems.

The first system utilizes a conduit, such as conduit 603, in operable connection with the sealed portion of the orifice such as the ear canal. In operable connection with this conduit is a pressure relief valve 801 which is configured as a positive and negative pressure “blow off valve” with a manual release. In the instance where the device is configured to be used in operable connection with the human ear canal and IBS 200 is in its occluding state, pressure relief valve 801 static default state is sealed and it enables flow in the forward or reverse direction at an ideal minimum pressure differential of approximately 3.0-10.0 kPa. Additionally, when the user desires to remove the device or it is removed inadvertently, pressure release valve 801 is preferably configured such that removal forces such as tension force 803 and sheer force 802 act against the relatively fixed IBS 200 causing pressure release valve 801 to open resulting in pressure release valve gap 804 enabling flow in both directions thereby mitigating any pressure differential between sealed portion of orifice and the ambient field. As removal forces may come from various directions, it may be advantageous to include several pressure release valve 801 as depicted in FIG. 20 so that any applied removal force will result in the opening of at least one pressure release valve 801. As will be obvious to those skilled in the art and depicted in FIGS. 18, 19, and 20, pressure relief valve 801 may be formed as a slit valve.

The second system depicted in FIGS. 18, 19, and 20 for pressure relief within the sealed orifice utilizes a conduit such as conduit 603 in operable connection with the sealed portion of the orifice such as the ear canal and CSP 610. As previously described, CSP 610 may include protruding portion 611 which is ideally configured to seal with conduit 603 in its static state. In the instance when forces such as tension force 803 and sheer force 802 are applied with the CSP—actuated orifice inserted inflated device 100c installed in the orifice and IBS 200 in its occluding state, the forces cause protruding portion 611 to be disconnected from conduit 603 resulting in the formation of CSP—conduit gap 805 which enables the displacement of any pressure differential between the ambient field and the sealed orifice. Preferably the aforementioned system will be configured such that forces such as tension force 803 and sheer force 802 result in the formation of CSP-Conduit gap 805 and thus pressure relief when the pressure differential created by the application of external forces on the device is equal to or greater than approximately 3.0-10.0 kPa.

FIGS. 21, 22 and 23 depict an alternate non-limiting embodiment of the present invention whereby the present invention is coupled with a pre-existing, universal CSP via standard interface. Referring to FIG. 23, this embodiment, universal CSP 900 is coupled to universal CSP orifice inserted device 100d. Universal CSP 900 may take many forms and is generally described as any pre-existing CSP which is designed to be in operable connection with an orifice via an independent device which is coupled to the CSP via a standard interface. An example of such a device is universal CSP 900 depicted in FIG. 23 which is representative of an ear bud. As will be obvious to those skilled in the art, universal CSP 900 is designed to interface with the human ear canal with tips such as foam tips that utilize a standard interface to couple to the CSP or headphone body. In this example, universal CSP orifice inserted device 100d is configured with standard connection 901 to be coupled to standard connector 901a of universal CSP 900 which includes universal CSP—orifice conduit 902 which makes CSP in operable connection with the orifice or ear canal.

The embodiment depicted in FIG. 21 is the inactivated default state of universal CSP orifice inserted device 100d. Universal CSP orifice inserted device 100d posses the same subsystems of IBS, FFMS, and FRS as the other aforementioned embodiments, however, they are adapted such that they can fit and operate in the size envelope equivalent to the device it is configured to replace. As is obvious in light of the figures, IBS 200 is an operational equivalent to IBS 200a with balloon system 204c an operational equivalent of balloon system 204. IBS 200a is in operable connection with FFMS 201b via conduit 206a which is an operational equivalent to conduit 206. FFMS 201b is an operational equivalent to FFMS 201 with flow regulation device 903 an operational equivalent to flow regulation device 207. FFMS 201b is in operable connection with FRS 202a which is an operational equivalent of FRS 202. FRS 202a is comprised of FRS compliant membrane 208b and FRS non-compliant liner 208c which function as an operational equivalent to FRS elastomeric membrane 208. IBS 200a, FFMS 201b, and FRS 202a are connected by CSP universal device body 904 which attaches to a CSP via standard connection 901 and includes universal CSP—orifice conduit 902 putting CSP in operable connection with occluded orifice such as an ear canal.

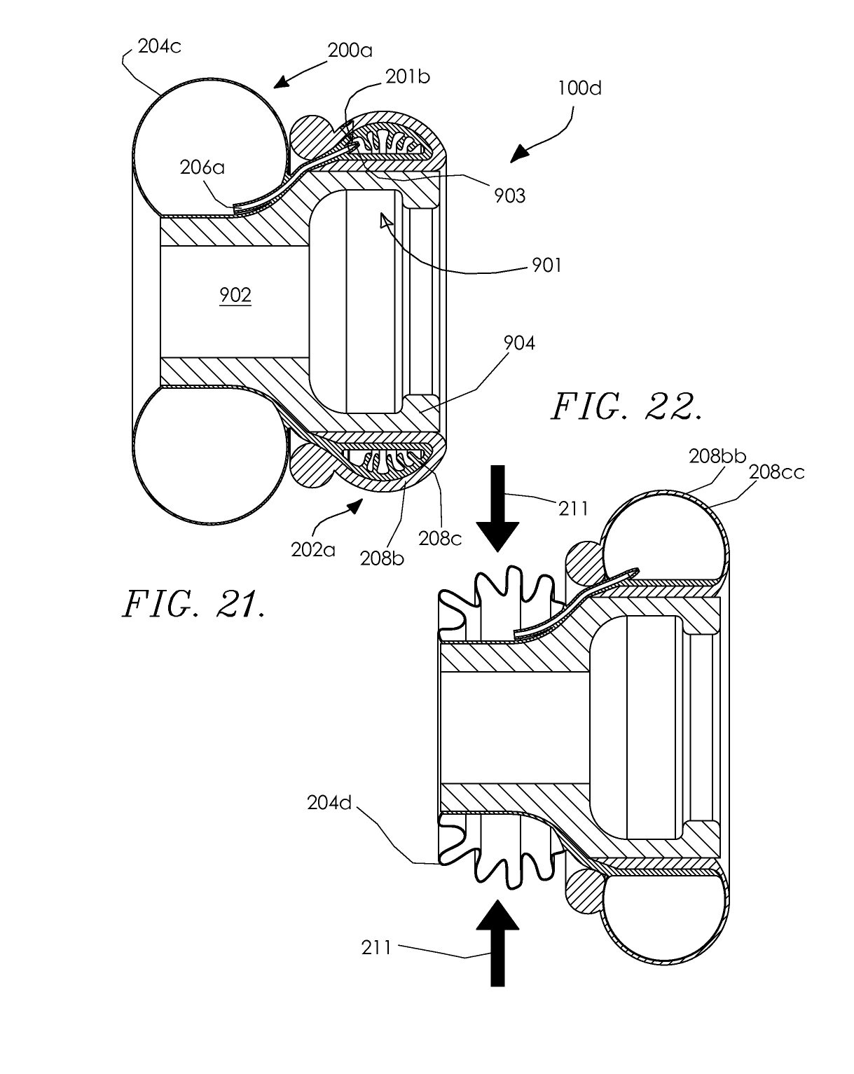

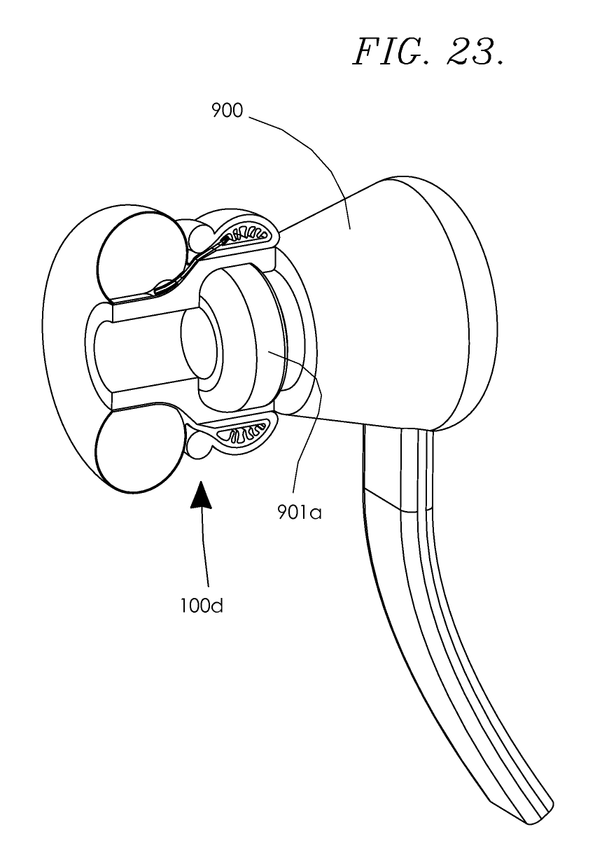

The embodiment depicted in FIG. 22 is the deflated/activated state of universal CSP orifice inserted device 100d. The process for achieving deflation, insertion into the orifice and orifice occlusion, is accomplished in an identical fashion to that of orifice inserted device 100a with the user applying IBS deflation force 211 which increases the pressure within IBS 200a causing fluid to flow through conduit 206a and FFMS 201b into FRS 202a resulting in the deflation of balloon system 204c into deflated balloon system 204d and the inflation of FRS compliant membrane 208b and FRS non-compliant liner 208c into inflated FRS compliant membrane 208bb and FRS non-compliant liner 208cc and an increase in FRS 202a internal pressure. Upon removal of IBS deflation force 211 the user inserts the device into the appropriate orifice and the higher pressure in FRS 202a in relation to IBS 200a causes fluid to flow back into IBS 200a, regulated by FFMS 201b, thus inflating balloon system 204c until it contacts the walls of the orifice, preferably creating an occluding seal, and achieving an internal pressure equivalent to that of FRS 202a.

What is claimed is:

1. An orifice occluding device comprising:
an inflatable balloon system comprises at a minimum a balloon operable to inflate and deflate upon the insertion and removal of fluid;
a fluid reservoir system in fluid communication with the inflatable balloon system operable to receive fluid and store it under pressure;
a fluid flow management system in fluid communication with both the inflatable balloon system and fluid reservoir system operable to regulate flow of fluid between said communicatively coupled systems.

2. The orifice occluding device according to claim 1, wherein:
the in the equilibrium uninstalled state of the device, the inflatable balloon system is in its fully inflated state, the fluid reservoir maintains positive fluid pressure within the device and no fluid flows through the fluid flow management system.

3. The orifice occluding device according to claim 1, wherein:
the inflatable balloon system is configured to be deflated by the application of compressive force onto the balloon of the inflatable balloon system causing fluid to be transferred through the fluid flow management system into the fluid reservoir system where it is stored under pressure and upon removal of said compressive force a fluid pressure differential exists between the inflatable balloon system and the fluid reservoir resulting in the flow of fluid from the fluid reservoir through the fluid flow management system into the inflatable balloon system.

4. The orifice occluding device according to claim 1, wherein:
the fluid flow management system is configured whereby it provides lower fluid flow resistance from the inflatable balloon system into the fluid reservoir system than flow from the fluid reservoir system into the inflatable balloon system.

5. The orifice occluding device according to claim 4, wherein:
the fluid flow management system is configured such that the resistance provided to fluid flow is decreased through the application of an external force onto the fluid flow management system.

6. The orifice occluding device according to claim 5, wherein:
fluid flow resistance provided by the fluid flow management system is modified through the application of force from a prime mover onto tissue in operable connection with the fluid flow management system whereby flow resistance modifying force is transferred through said tissue to the fluid flow management system.

7. The orifice occluding device according to claim 1, wherein:
a fluid vent exists in the device whereby the inner portion of the orifice occluded by the inflatable balloon system in fluidic commutation with the outer portion of the occluded orifice.

8. The orifice occluding device according to claim 7 wherein:
a valve resists flow until a pressure differential greater than approximately 3.0-10.0 kPa. exists between the occluded and outer portions of the orifice.

9. The orifice occluding device according to claim 8 wherein:
the valve is configured such that resistance to flow is removed upon the application of an extracting force to the device.

10. The orifice occluding device according to claim 1 wherein:
the device is configured to be coupled to a complementary systems package whereby the occluded orifice may be communicatively coupled with the complementary systems package.

11. The orifice occluding device according to claim 1 wherein:
the device is configured such that a compressible material such as foam is applied to the exterior of the device in areas which may contact adjacent tissue other than the inflatable balloon.
